(19) United States
(12) Patent Application Publication
(10) Pub. No.: US 2019/0164200 A1
(43) Pub. Date: May 30, 2019

(54) EYEGLASSES-MATCHING TOOL

(71) Applicant: ESSILOR INTERNATIONAL, Charenton-Le-Pont (FR)

(72) Inventors: Michel PROULX, Vancouver, BC (CA); Nicole OLIVER, Vancouver, BC (CA); Asim MASOOD, Vancouver, BC (CA); Scott MACGREGOR, Vancouver, BC (CA); Payam SHALCHIAN, Vancouver, BC (CA); Alisha KASSAM, Vancouver, BC (CA); Justin HSIEH, Vancouver, BC (CA)

(21) Appl. No.: 16/321,187

(22) PCT Filed: Jul. 28, 2017
(86) PCT No.: PCT/IB2017/000887

§ 371 Date: Jan. 28, 2019

Related U.S. Application Data
(60) Provisional application No. 62/367,991, filed on Jul. 28, 2016.

(51) Int. Cl. G06Q 30/02; G02C 7/02; A61B 3/04

(52) U.S. Cl. CPC G06Q 30/0281; G02C 13/003; A61B 3/04; G02C 7/027

(57) ABSTRACT

An eyeglasses-matching tool may provide consumers with a high level of confidence that eyeglasses purchased online will have a proper fit. The consumer provides frame measurements from the consumer's current eyeglasses. The frame measurements may include a lens width, bridge width, and temple arm length, such as typically found printed on the inside temple arm of eyeglasses. The eyeglasses-matching tool may recommend eyewear styles and products that have similar measurements and may provide contextual information regarding eyewear styles, such as a relative fit compared to the consumer's current eyeglasses.

### CROSS-REFERENCE TO RELATED APPLICATIONS

This application claims the benefit of the filing date of U.S. Provisional Patent Application No. 62/367,991 which is also entitled “Eyeglasses-Matching Tool” and which was filed Jul. 28, 2016, the disclosure of which is hereby incorporated by reference herein in its entirety.

### TECHNICAL FIELD

The present disclosure relates generally to eyeglasses frame fitting, more particularly to eyeglasses frame matching tools, and specifically to online eyeglasses frame matching tool systems and methods.

### BACKGROUND

Currently, the on-line eyewear industry accounts for just 4% of the market for customers purchasing prescription eyewear. Many consumers lack confidence in prescription eyewear purchased through online channels because they are not sure that products shown online will fit them. As a result, 96% of consumers purchase prescription eyewear from brick and mortar stores where they can try on eyeglasses frames to evaluate the fit.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

The present invention is directed to systems and methods which provide an eyeglasses-matching tool that affords consumers a high level of confidence that eyeglasses purchased online will have a proper fit. The consumer provides the frame measurements from one or more of the consumer's current eyeglasses frame. The frame measurements may include a lens width, bridge width, and temple arm length, such as typically found printed on the inside temple arm of eyeglasses frame.

The eyeglasses-matching tool may recommend eyewear styles and products that have similar measurements to the consumer's current eyeglasses frame. As used herein, an eyeglasses frame “style” is an eyeglasses model from a particularly eyeglasses frame brand, or the like. The eyeglasses-matching tool may provide contextual information regarding eyewear styles, such as a relative fit compared to the consumer's current eyeglasses frame.

In various embodiments, one or more of the techniques described herein may be performed by one or more computer systems. In other various embodiments, a tangible computer-readable storage medium may have program instructions stored thereon that, upon execution by one or more computer systems, cause the one or more computer systems to execute one or more operations disclosed herein. In yet other various embodiments, one or more systems may each include at least one processor and memory coupled to the processor(s), wherein the memory is configured to store program instructions executable by the processor(s) to cause the system(s) to execute one or more operations disclosed herein.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following, description when considered in connection with the accompanying figures. It is to be expressly understood, however, that each of the figures is provided for the purpose of illustration and description only and is not intended as a definition of the limits of the present invention.

### DRAWINGS

To further clarify the above and other advantages and features of embodiments of the present invention, a more particular description of embodiments of the present invention will be rendered by reference to the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a frame size code on an eyeglasses frame temple arm;

FIGS. 2A and 2B illustrate the frame size measurements on eyeglasses frames;

FIG. 3 is a flowchart of an example process for eyeglasses frame matching, according to some embodiments;

FIG. 4 is a block diagram illustrating an example system for providing an online eyeglasses-matching tool, according to some embodiments; and

FIGS. 5 through 14 are example user interfaces for an eyeglasses-matching tool.

While this specification provides several embodiments and illustrative drawings, a person of ordinary skill in the art will recognize that the present specification is not limited only to the embodiments or drawings described. It should be understood that the drawings and detailed description are not intended to limit the, specification to the particular form disclosed, but, on the contrary, the intention is to cover all modifications, equivalents and alternatives falling within the spirit and scope of the claims. As used herein, the word “may” is meant to convey a permissive sense (i.e., meaning “having the potential to”), rather than a mandatory sense (i.e., meaning “must”). Similarly, the words “include,” “including,” and “includes” mean “including, but not limited to.”

### DETAILED DESCRIPTION

The invention now will be described more fully hereinafter with reference to the accompanying drawings. This invention may, however, be embodied in many different forms and should not be construed as limited to the embodiments set forth herein. Rather, these embodiments are provided so that this disclosure will be thorough and complete, and will fully convey the scope of the invention to those skilled in the art. One skilled in the art may be able to use the various embodiments of the invention.

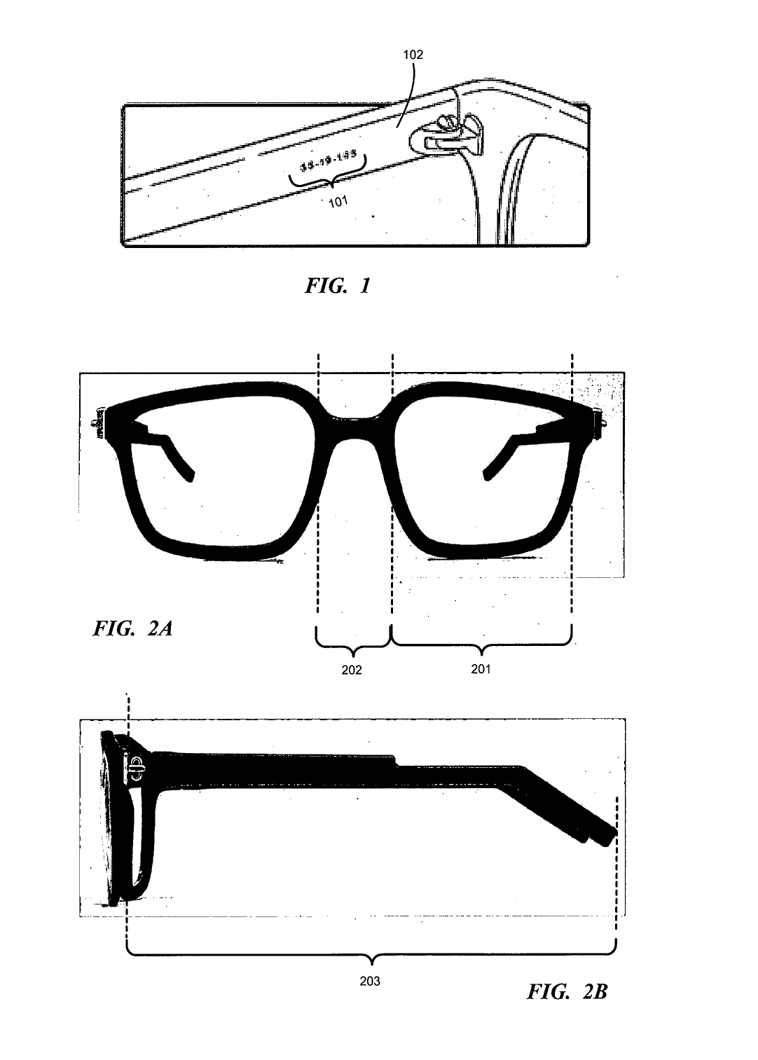

An eyeglasses-matching online tool can be used to help consumers find eyeglasses with the proper fit. The eyeglasses-matching tool disclosed herein gives consumers confidence that they can find eyeglasses frames with a proper fit, such as eyewear that will sit correctly on a consumer's face. As shown in FIG. 1, most eyeglasses frames carry a standard mark 101 on the inside of the left temple arm 102. The mark 101 comprises three measurements: lens width, bridge width, and temple arm length, usually expressed in millimeters.

These measurements are indicated in FIGS. 2A and 2B. The lens width 201 is the width of one lens measured in millimeters. This number is relevant to a frame's overall width. Bridge width 202 is the second number in the string and is also a factor in determining overall width. The bridge width measurement 202 is the distance between the lenses. The lens width 201 and bridge width 202 may for different frames vary a relatively significant amount, depending upon the eyeglasses frame style and impact how well the frame will fit the consumer's face. The third number in the string is the temple length 203. This is the measurement in millimeters of the arm or earpiece of the frame. The temple length measurement 203 typically does not vary as much as the lens width 201 and bridge width 202 across different eyeglasses frame styles and is often set to 135 mm, 140 mm, 145 mm or 150 mm. In accordance with various embodiments of the present systems and methods, an online eyeglasses-matching tool prompts customers to enter these measurements from a current pair of eyeglasses. Using this information, the online eyeglasses-matching tool can select available products that are compatible with the customer's current eyewear measurements.

The online eyeglasses-matching tool may function as a starting point in online eyewear shopping by recommending frames that relate to the customer's current eyeglasses frame size. Unlike existing eyewear sites, the online eyeglasses-matching tool does more than merely filter or sort eyewear. Instead, the online eyeglasses-matching tool provides a framework to tailor eyewear product content and results to each individual customer at any point in their online shopping experience.

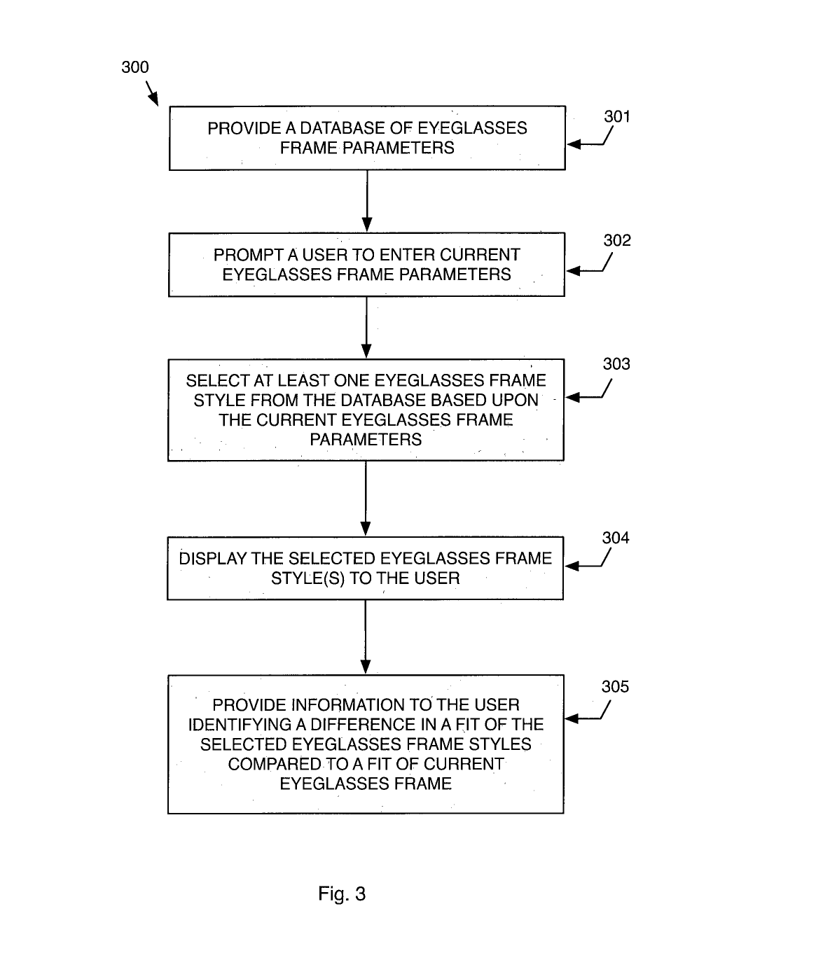

FIG. 3 is a flowchart of process 300 for eyeglasses frame matching, according to some embodiments. Therein, a database of eyeglasses frame parameters is provided at 301. At 302 a user is prompted, such as via an online interface to enter current eyeglasses frame parameters, such as eyeglasses frame measurements of the user's current eyeglasses, such as may be printed on the inside of the left temple arm of the user's current eyeglasses frame, for example. These current eyeglasses frame measurements may comprise a lens width, a bridge width, and a temple length and may be provided, in accordance with some embodiments of the present systems and methods from more than one pair of eyeglasses.

At least one eyeglasses frame styles are selected by the eyeglasses-matching tool from the database at 303, based upon the current eyeglasses frame parameters entered at 302. More specifically, in accordance with some embodiments of the present systems and methods, the online eyeglasses-matching tool will not present the user with eyewear products that will not fit the customer based on the current eyewear measurements. By presenting only eyeglasses frames that are within range of the customer's current eyeglasses, the risk of buying ill-fitting eyewear is reduced and the consumers will have confidence in their choice even though the eyeglasses frame are selected online and not in person.

However, the present online eyeglasses-matching tool does not have'to limit the eyewear that is displayed to the customer to exact matches of the customer's current measurements. Instead, the online eyeglasses-matching tool may select eyewear having measurements that are within a predetermined variation of the customer's current eyewear measurements. To wit, the selection of eyeglasses frame style(s) at 303 may entail embodiments of the present systems and methods calculating an acceptable variation in the current eyeglasses frame parameters, such as the entered measurements and selecting the eyeglasses frame style(s) based upon the acceptable variation in the current eyeglasses frame parameters. For example, the tool may select eyewear products that have a lens width measurement that differs from the customer's current eyeglasses by a certain number of millimeters or a certain percentage as long as the overall frame width is similar.

The present online eyeglasses-matching tool offers a more contextualized experience than existing online eyewear retailers. For example, the eyeglasses frame style(s) selected by the eyeglasses-matching tool at 303 are displayed to the user, such as via the online interface, at 304. At 305, information is provided to the user identifying a difference in a fit of the selected eyeglasses frame style(s) compared to a fit of the current eyeglasses frame. This difference in the fit of the selected eyeglasses frame style(s) may comprise a difference in how the eyeglasses frame fit on a user's face. Additionally, or alternatively, the difference in the fit of the selected eyeglasses frame style(s) may comprise a difference in the frame's shape.

In further embodiments, the eyeglasses-matching tool may compare multiple sets of measurements for a single customer. Based on the customer's previously purchased or current pair of eyeglasses, the eyeglasses-matching tool can determine acceptable variations in frame size for that customer. The determined variations can be used to select which products should be displayed for the customer.

Additionally, the eyeglasses-matching tool can provide contextual comparisons to other eyewear frames that customer is browsing. For example, the eyeglasses-matching tool may provide feedback, such as “this frame is wider on your face than your current glasses” or “this frame sits higher on your nose than your current glasses”. This feedback allows customers to use their current pair of eyeglasses as a reference point to understand how a pair of frames will fit their face.

As a further example of the present, online eyeglasses-matching tool offering a more contextualized experience than existing online eyewear retailers, the customer can select the online eyeglasses-matching tool while viewing a particular eyewear product and the tool will validate and compares the fit of the selected product with the customer's current frames. For example, the parameters of the particular eyewear product the customer is viewing may be retrieved from a database of eyeglasses frame parameters and compared to the customer's current eyeglasses frame parameters. These current customer eyeglasses frame parameters may have been previously entered, or the customer may be prompted to enter their current eyeglasses frame parameters upon selection of the online eyeglasses-matching tool.

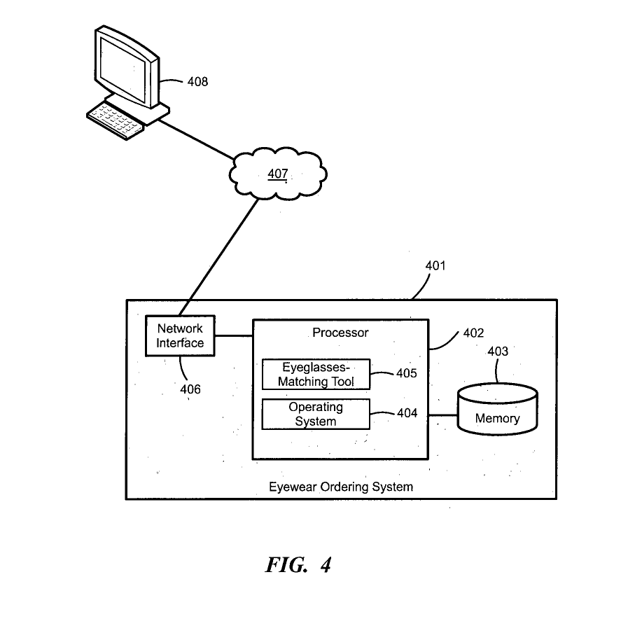

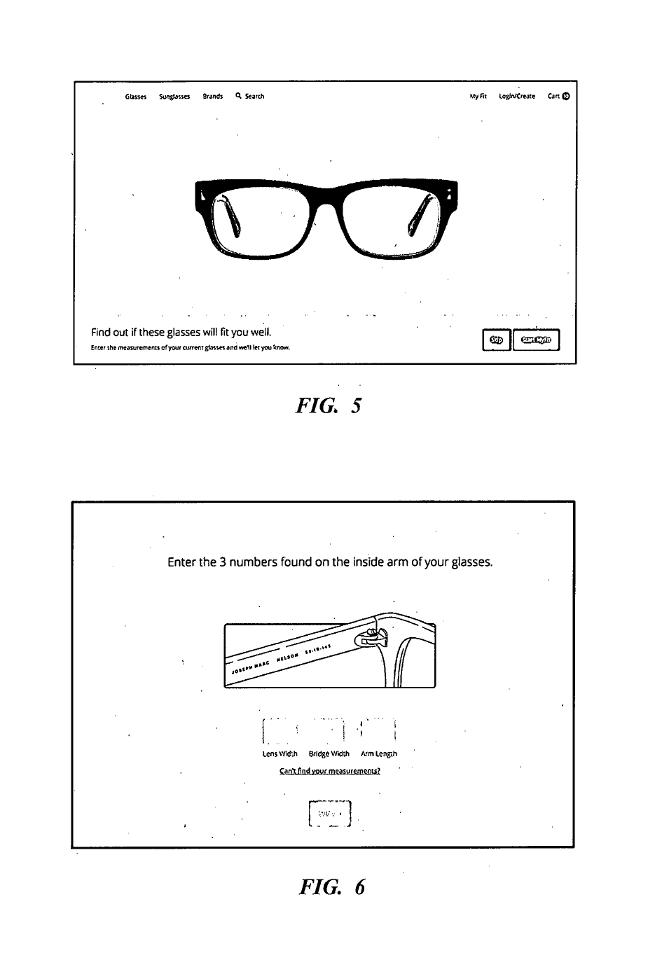

FIG. 4 is an example system for providing an online eyeglasses-matching tool. An eyewear ordering system 401, such as computer server, comprises a processor 402 and a memory 403, which stores software instructions for execution on processor 402. For example, the software instructions may comprise operating system code 404 and eyeglasses-matching tool application code 405.

Network interface 406 provides access to eyewear ordering system 401 via network 407, which may include any public or private network, such as the Internet. Users access eyewear ordering system 401 via terminal 408, which may be, for example, a kiosk, desktop computer, laptop, tablet, smart phone, or other device, and which may provide a user interface, or the like.

The eyeglasses-matching tool 405 may be presented to users on terminal 408 as a webpage, providing a user interface. The users may access the eyeglasses-matching tool 405 via a web browser that is pointed to a URI for the eyewear ordering system server 401.

A person of ordinary skill in the art will appreciate the system of FIG. 4 is merely illustrative and is not intended to limit the scope of the disclosure described herein. In particular, a computer system and devices implementing the present eyeglasses-matching tool may include any combination of hardware or software that can perform the indicated operations. Additionally, the operations performed by the illustrated components may, in some embodiments, be performed by fewer components or distributed across additional components. Similarly, in other embodiments, the operations of some of the illustrated components may not be provided and/or other additional operations may be available. Accordingly, systems and methods described herein may be implemented or executed with other computer system configurations.

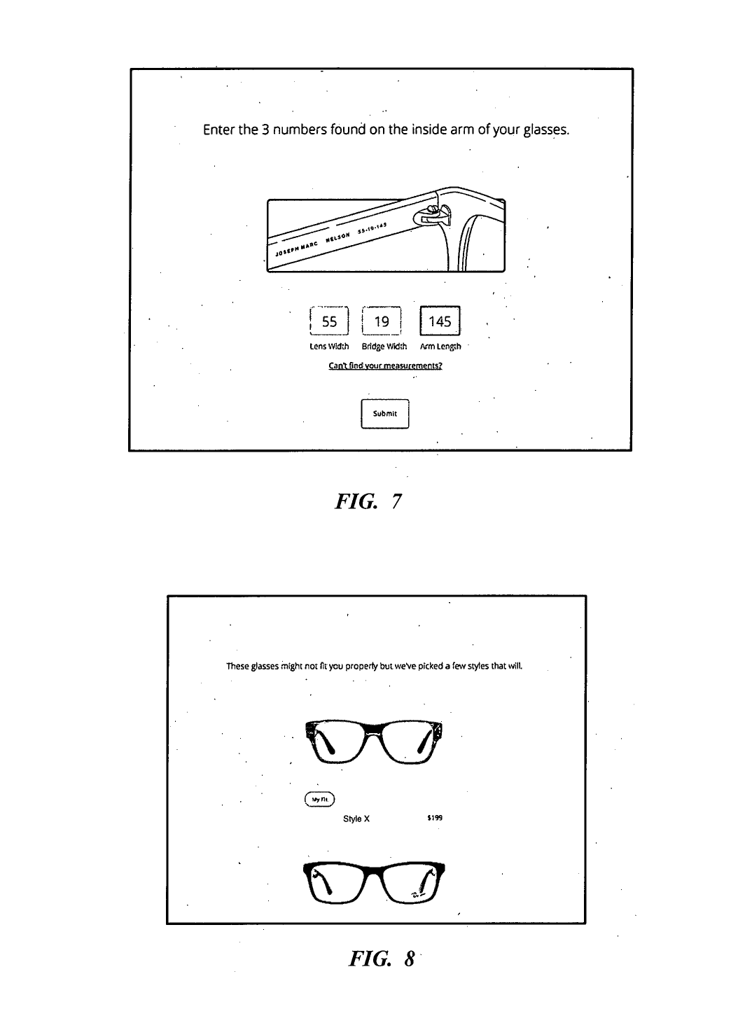

FIGS. 5 through 15 are example user interface screens that may be presented to a user to access the eyeglasses-matching tool, in accordance with various embodiments of the present systems and methods. For example, FIG. 5 is a screen displaying a particular set of frames that a customer is viewing. This screen offers the customer an option to launch the present eyeglasses-matching tool to “find out if these glasses will fit you well.” FIG. 6 shows a screen where a customer can enter their current eyeglasses frame parameters, such as may be displayed upon launching the current, eyeglasses-matching tool. FIG. 7 shows the screen of FIG. 6, wherein the customers current eyeglasses frame parameters have been entered. The screens of FIGS. 6 and 7 each offer the customer assistance in finding the current eyeglasses frame parameters by providing a link marked, “Can't find your measurements?”

After comparing the frames being viewed by the customer with the customers current eyeglasses frame parameters, a screen such as shown in FIG. 8 may indicate whether the particular frames being viewed would fit properly and may, as also shown, display suggested styles selected by the present eyeglasses-matching tool, such as may be selected in a manner similar to step 303 discussed above.

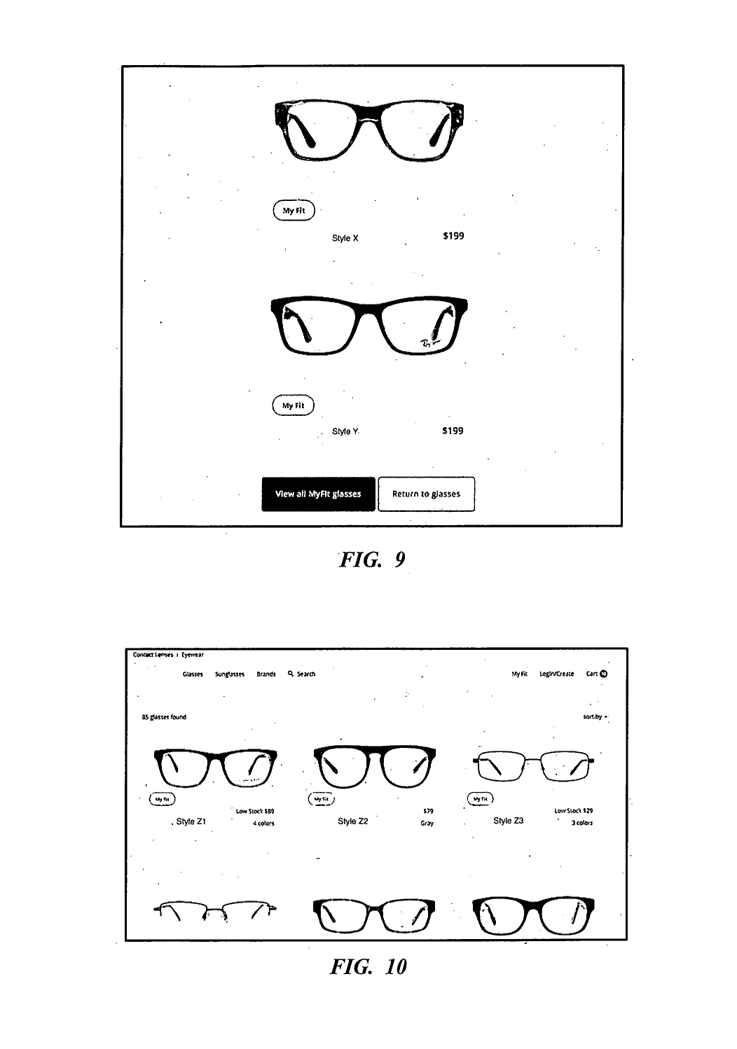

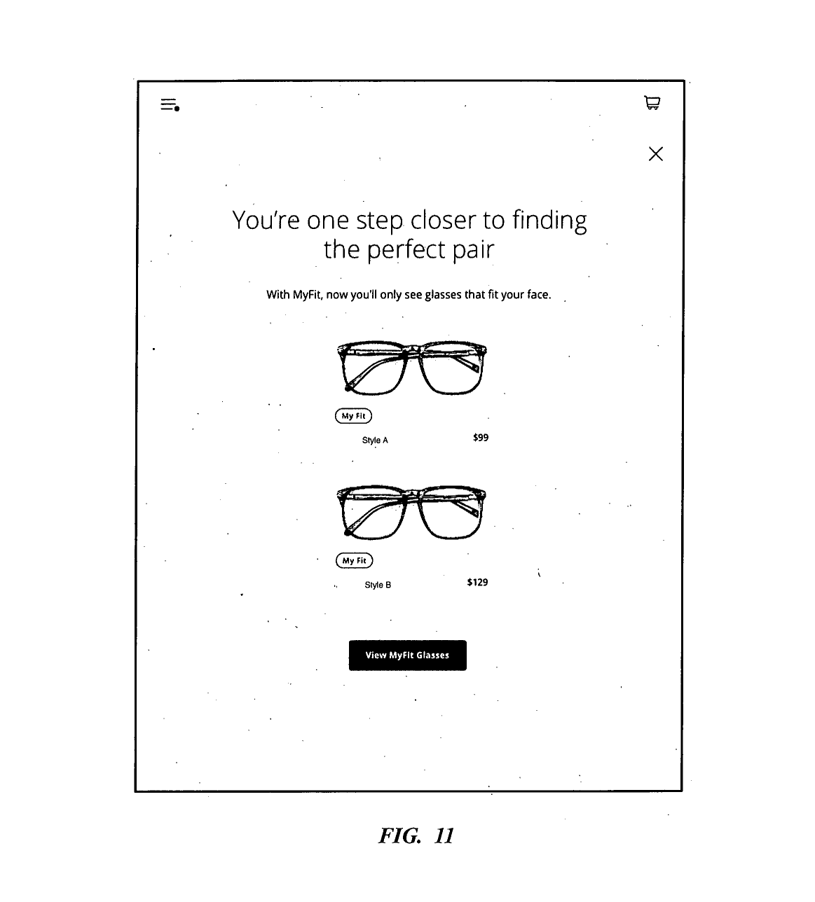

While viewing eyeglasses frames, as shown in FIG. 9, a user my select to “View all glasses that fit me,” or a similar provided selection, to view all frames that the present eyeglasses-matching tool may select based upon the current eyeglasses frame parameters entered, such as may be selected in a manner similar to step 303 discussed above. The screen of FIG. 10 displays the eyeglasses-matching tool selected eyeglasses frame styles, such as in a manner similar to step 304 discussed above.

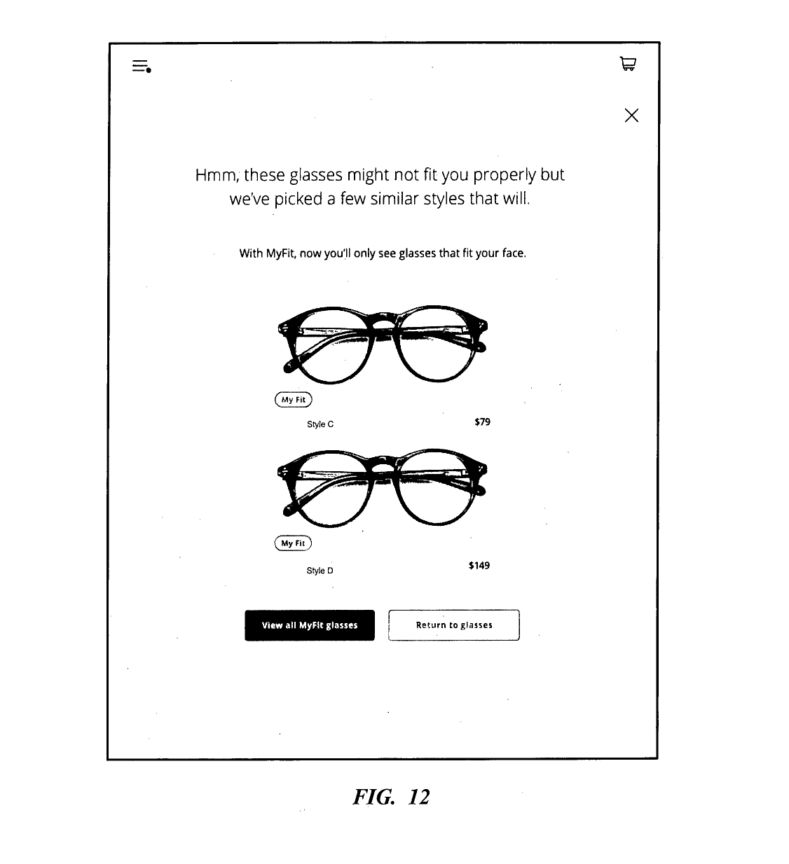

Similar to FIG. 5, FIG. 11 is a screen displaying two particular sets of frames that a customer is viewing. This screen offers the customer an option to launch the present eyeglasses-matching tool. Again, after comparing the frames being viewed by the customer with the customers current eyeglasses frame parameters, a screen such as shown in FIG. 12 may indicate whether the particular frames being viewed would fit properly and may, as also shown, display suggested styles selected by the present eyeglasses-matching tool, such as may be selected in a manner similar to step 303 discussed above. In this case, the alternative frames shown are of a similar style to the frames being viewed by the customer when the eyeglasses-matching tool was launched.

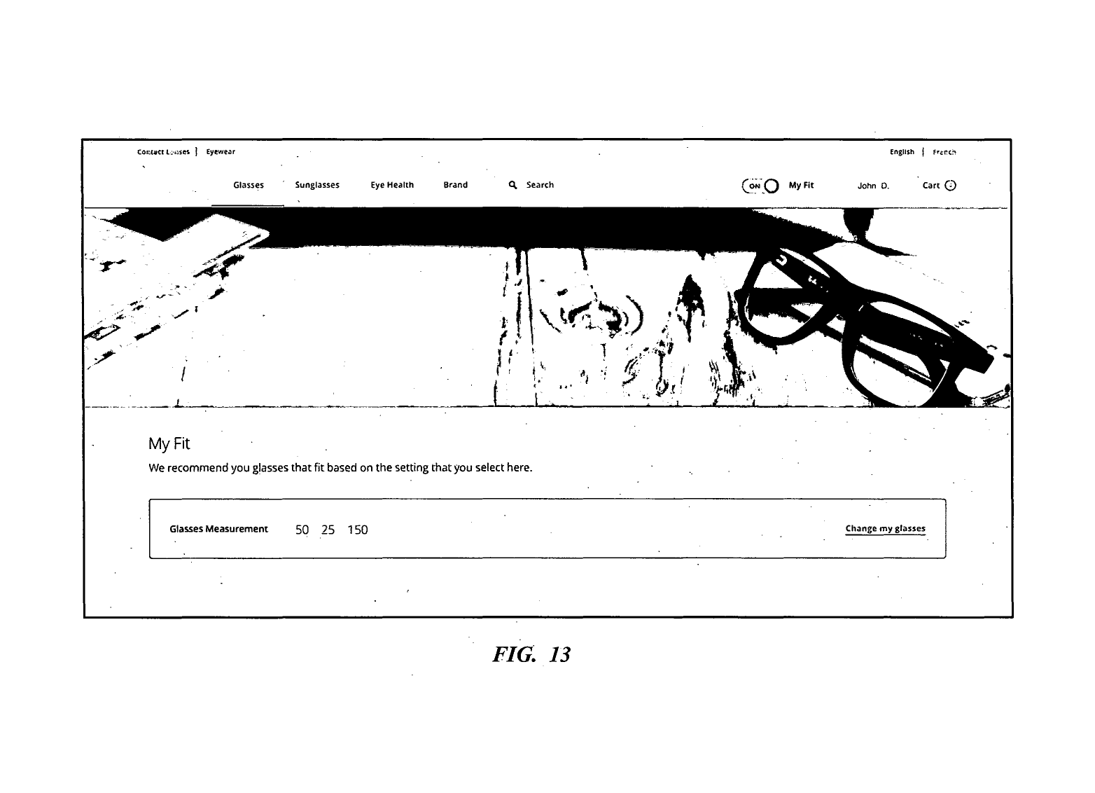

FIG. 13 is an eyeglasses-matching tool “home page” for a customer employing the present systems and methods where, for example customer current eyeglasses frame parameters may be stored and changed and links may be provided associated with previously purchased frames, or the like.

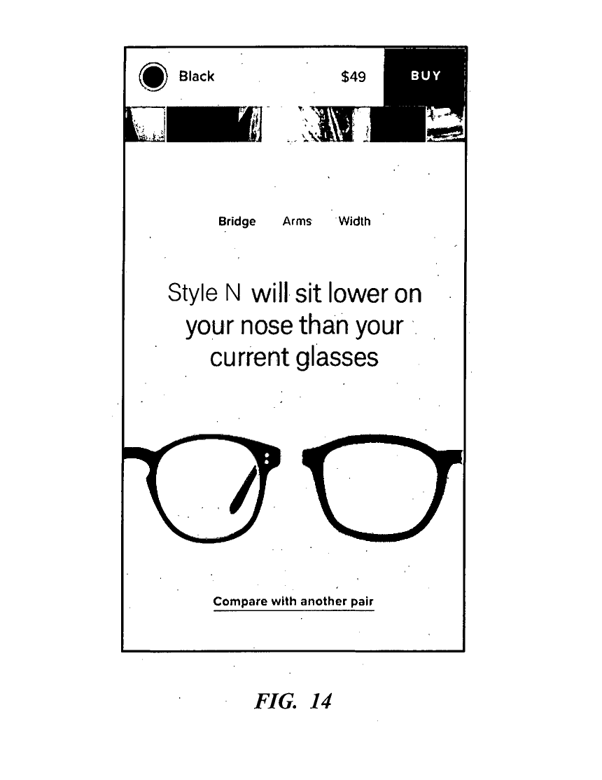

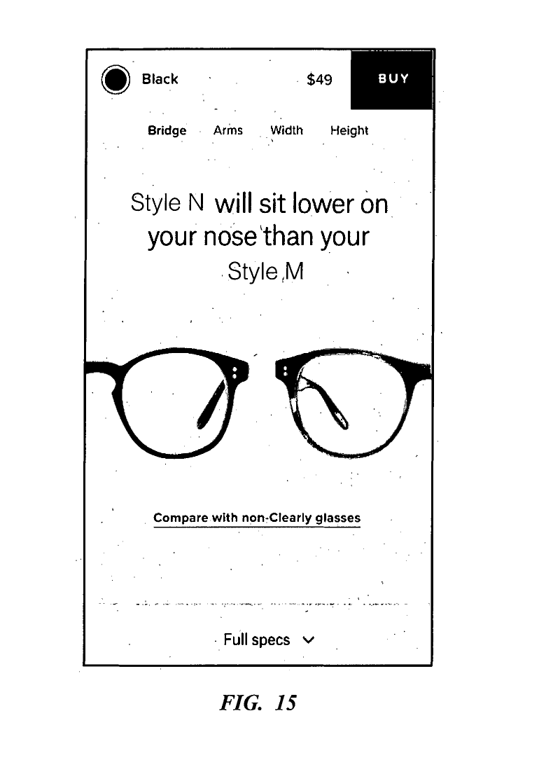

FIG. 14 shows a screen which provides information to, the user identifying a difference in a fit of the selected eyeglasses frame styles compared to a fit of current eyeglasses frame, such as in accordance with step 305 discussed above, based on the current eyeglasses frame parameters. Similarly, FIG. 15 shows a screen which provides information to the user identifying a difference in a fit of the selected eyeglasses frame styles compared to a fit of the customer's current eyeglasses frame, based on the current eyeglasses frame style, such as may have been retained by the eyeglasses-matching tool for that particular customer.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method (300), comprising:
providing (301) a database of eyeglasses frame parameters;
prompting (302) a user to enter current eyeglasses frame parameters;
selecting at least one eyeglasses frame style from the database based upon the current eyeglasses frame parameters;
displaying (303) the selected at least one eyeglasses frame style to the user; and
providing (304) information to the user identifying a difference in a fit of the selected at least one eyeglasses frame style compared to a fit of the current eyeglasses frame.

2. The method of claim 1, wherein the current eyeglasses frame parameters comprise current eyeglasses frame measurements (101).

3. The method of claim 2, wherein the current eyeglasses frame measurements comprise a lens width, a bridge width, and a temple length (201 through 203).

4. The method of claim 1, further comprising:
comparing eyeglasses frame parameters of at least one eyeglasses frame style the user is viewing on an interface to the current eyeglasses frame parameters; and
providing information to the user as to fit of the at least one eyeglasses frame style being viewed compared to fit of the current eyeglasses frame.

5. The method of claim 1, further comprising:
calculating an acceptable variation in the current eyeglasses frame parameters; and
selecting the at least one eyeglasses frame style based upon the acceptable variation in the current eyeglasses frame parameters.

6. The method of claim 1, wherein the difference in the fit of the selected at least one eyeglasses frame style comprises a difference in how the eyeglasses frame fit on a user's face.

7. The method of claim 1, wherein the difference in the fit of the selected at least one eyeglasses frame style comprises a difference in the eyeglasses frame's shape.

8. An eyeglasses-matching tool (405), comprising:
a database of eyeglasses frame parameters (403);
a processor (402); and
a memory (403) coupled to the processor, the memory having program instructions stored thereon that, upon execution by the processor, cause the eyeglasses-matching tool to:
prompt a user to enter current eyeglasses frame parameters (302);
select at least one eyeglasses frame style from the database based upon the current eyeglasses frame parameters (303);
display the selected at least one eyeglasses frame style to the user on a user interface (304); and
provide information to the user, on the user interface, identifying a difference in a fit of the selected at least one eyeglasses frame style compared to a fit of the current eyeglasses frame (305).

9. The eyeglasses-matching tool of claim 8, wherein the current eyeglasses frame parameters comprise current eyeglasses frame measurements (101).

10. The eyeglasses-matching tool of claim 9, wherein the current eyeglasses frame measurements comprise a lens width, a bridge width, and a temple length (201, 202 and 203).

11. The eyeglasses-matching tool of claim 8, wherein the program instructions, upon execution, cause the eyeglasses-matching tool to:
compare eyeglasses frame parameters of at least one eyeglasses frame style the user is viewing on the user interface to the current eyeglasses frame parameters; and
provide information to the user, on the user interface, as to fit of the at least one eyeglasses frame style being viewed compared to fit of the current eyeglasses frame.

12. The eyeglasses-matching tool of claim 8, wherein the program instructions, upon execution, cause the eyeglasses-matching tool to:
calculate an acceptable variation in the current eyeglasses frame parameters; and
select the at least one eyeglasses frame style based upon the acceptable variation in the current eyeglasses frame parameters.

13. The eyeglasses-matching tool of claim 1, wherein the difference in the fit of the selected at least one eyeglasses frame style comprises a difference in how the eyeglasses frame fit on a user's face.

14. The eyeglasses-matching tool of claim 1, wherein the difference in the fit of the selected at least one eyeglasses frame style comprises a difference in the eyeglasses frame's shape.
